(19) United States
(12) Patent Application Publication
(10) Pub. No.: US 2025/0135160 A1
(43) Pub. Date: May 1, 2025

(54) INFLOW / OUTFLOW CANNULA ANCHORS

(71) Applicant: W. L. Gore & Associates, Inc., Newark, DE (US)

(72) Inventors: Steven M. Alston, JR., Flagstaff, AZ (US); Scott M. Bryson, Newark, DE (US); Daniel Dominguez, Anthem, AZ (US); James L. Goepfrich, Flagstaff, AZ (US); Joshua C. Haarer, Flagstaff, AZ (US); Edward E. Shaw, Flagstaff, AZ (US)

(21) Appl. No.: 18/699,048

(22) PCT Filed: Oct. 5, 2022
(86) PCT No.: PCT/US2022/045773

§ 371 Date: Apr. 5, 2024

Related U.S. Application Data
(60) Provisional application No. 63/253,391, filed on Oct. 7, 2021.

(51) Int. Cl. A61M 25/04; A61M 25/02

(52) U.S. Cl. CPC A61M 25/04; A61M 2025/0293

(57) ABSTRACT

Various aspects of the present disclosure are directed toward apparatuses, systems, and methods that may include an inflow or outflow cannula apparatus.

### CROSS-REFERENCE TO RELATED APPLICATION

This application is a national phase application of PCT Application No. PCT/US2022/045773, internationally filed on Oct. 5, 2022, which claims the benefit of Provisional Application No. 63/253,391, filed Oct. 7, 2021, which are incorporated herein by reference in their entireties for all purposes.

### FIELD

This disclosure relates generally to medical devices, and more specifically, to inflow or outflow cannulas that may include a tissue anchor.

### BACKGROUND

Cannulas may be able to create flow conduits within a patient. For example, cannulas may be used to create flow conduits to or from an organ such as the heart. U.S. Pat. No. 10,849,653 to Tao et al., describes a cannula supporting a percutaneous pump that includes a proximal section with a first flexural modulus and one or more distal sections with a flexural modulus that is different than the first flexural modulus. The material and its arrangement along the length of the cannula can be selected so as to influence bending properties. This can, for example, allow efficient positioning of the cannula in a desired location without displacing the guidewire. Though Tao provides an example of one use, cannulas may be used in other contexts, such as for interconnecting or bypassing portions of the vasculature (e.g., between one or more vessels).

### SUMMARY

According to one example (“Example 1”), a cannula includes a conduit having an exterior surface, an interior surface, a first end, and a second end; and an anchoring structure arranged at the first end of the conduit, the anchoring structure including: a tubular body including a first end, a second end, and a lumen configured to receive the end of the conduit within the tubular body, a first plurality of struts extending from a first longitudinal position between the first and second ends of the tubular body or from adjacent the second end of the tubular body toward the first end of the tubular body with each of the first plurality of struts including a first pad, and a second plurality of struts of struts extending from a second longitudinal position that is the same or different from the first longitudinal position and is between the first and second ends of the tubular body or extending from adjacent the first end of the tubular body toward the second end of the tubular body with each of the second plurality of struts including a second pad wherein the first and second plurality of struts cross one another longitudinally.

According to another example (“Example 2”), the cannula of Example 1, the first plurality of struts extends from adjacent the second end of the tubular body toward the first end of the tubular body and the second plurality of struts extend from a midpoint of the tubular body.

According to another example (“Example 3”), the cannula of any one of Examples 1-2, the first pad of each of the first plurality of struts extends a first distance beyond the first end of the tubular body and the second pad of each of the second plurality of struts extends a second distance that is greater than the first distance beyond the second end of the tubular body in a delivery configuration.

According to another example (“Example 4”), the cannula of any one of Examples 1-3, the first plurality of struts are configured to extend longitudinally from the tubular body and toward the second end of the tubular body and the second plurality of struts are configured to extend longitudinally from the tubular body and toward the first end of the tubular body in a deployed configuration.

According to another example (“Example 5”), the cannula of Example 4, the first plurality of struts are longitudinally spaced from the second plurality of struts along a longitudinal axis of the tubular body in the deployed configuration.

According to another example (“Example 6”), the cannula of any one of Examples 4-5, the first pad of each of the first plurality of struts and the second pad of each of the second plurality of struts are configured to contact opposing surfaces of a tissue wall in the deployed configuration.

According to another example (“Example 7”), the cannula of any one of the preceding Examples, the first plurality of struts are circumferentially staggered relative to the second plurality of struts along the tubular body.

According to another example (“Example 8”), the cannula of any one of the preceding Examples, the first pad of each of the first plurality of struts is operable to longitudinally transition toward the second end of the tubular body and the second pad of each of the second plurality of struts is operable to longitudinally transition toward the first end of the tubular body when the anchoring structure is transitioned toward the deployed configuration.

According to another example (“Example 9”), the cannula of any one of the preceding Examples, wherein the first pad of each of the first plurality of struts and the second pad of each of the second plurality of struts is operable to transition radially outward when the anchoring structure is transitioned toward the deployed configuration.

According to another example (“Example 10”), the cannula of any one of the preceding Examples, the first plurality of struts and the second plurality of struts include a curved profile when in the deployed configuration.

According to another example (“Example 11”), the cannula of Example 10 the curved profile includes a first portion that is curved in a first direction and a second portion that is curved in a second direction.

According to another example (“Example 12”), the cannula of Example 11, the first portion of the curved profile is greater than 90 degrees.

According to another example (“Example 13”), the cannula of any one of the preceding Examples, the first plurality of struts are operable to be deployed prior to the second plurality of struts.

According to another example (“Example 14”), the cannula of any one of the preceding Examples, the first plurality of struts and the second plurality of struts are shape set to the deployed configuration.

According to another example (“Example 15”), the cannula of any one of the preceding Examples, wherein the tubular body includes a wall including apertures formed through the wall.

According to another example (“Example 16”), the cannula of any one of the preceding Examples, the first end of the tubular body is fluted.

According to one example (“Example 17”) an anchoring structure of a cannula includes a tubular body having a wall including a first end and a second end, the wall having a lumen defined from the first end to the second; a first plurality of arms circumferentially spaced and extending radially outward from the tubular body at a first longitudinal position, each arm of the first plurality of arms including a base portion positioned within the lumen of the tubular body and a body portion extending through and beyond the wall of the tubular body; and a second plurality of arms circumferentially spaced and extending radially outward from the tubular body at a second longitudinal position, each arm of the second plurality of arms including a base portion positioned within the lumen of the tubular body and a body portion extending through and beyond the wall of the tubular body, the second plurality of arms being circumferentially offset relative to the first plurality of arms.

According to another example (“Example 18”), the anchoring structure of Example 17, the body portion of the first plurality of arms and the second plurality of arms is wider at a mid-portion than at radial ends of the first plurality of arms and the second plurality of arms.

According to another example (“Example 19”), the anchoring structure of either of Examples 17 or 18, the first plurality of arms and the second plurality of arms have a parallelogram shape.

According to another example (“Example 20”), the anchoring structure of any one of Examples 17-19, the base portion of the first plurality of arms and the second plurality of arms is shaped to conform to an inner surface of the wall of tubular body.

According to another example (“Example 21”), the anchoring structure of any one of Examples 17-20, the first end of the tubular body has first inner diameter and the second end of the tubular body has a second inner diameter, wherein the first inner diameter is greater than the second inner diameter.

According to another example (“Example 22”), the anchoring structure of any one of Examples 17-21, wherein the first plurality of arms and the second plurality of arms are wires.

According to one example (“Example 23”), an anchoring structure of a cannula includes a tubular body having a wall including a first end and a second end, the wall having a lumen defined from the first end to the second; a first longitudinal stop including a first plurality of arms circumferentially spaced and extending radially outward from a first ring engaged with the tubular body at a first longitudinal position; and a second longitudinal stop including a second plurality of arms circumferentially spaced and extending radially outward from a second ring engaged with the tubular body at a second longitudinal position, the second plurality of arms being circumferentially offset relative to the first plurality of arms.

According to another example (“Example 24”), the anchoring structure of Example 23, the first plurality of arms has a first arm length and the second plurality of arms has a second arm length, the first arm length being less than the second arm length.

According to another example (“Example 25”), the anchoring structure of any one of Examples 23-24, the each of the arms of the first plurality and second plurality of arms is wider at a tip than at a base.

According to another example (“Example 26”), the anchoring structure of any one of Examples 23-25, the tubular body includes a first circumferential slot formed in an outer surface of the wall and a second circumferential slot formed in an outer surface of the wall longitudinally spaced from the first circumferential slot, wherein the first ring is positioned in the first circumferential slot and the second ring is positioned in the second circumferential slot.

According to another example (“Example 27”), the anchoring structure of Example 26, the first and second longitudinal stops have a thickness and the first and second circumferential slots each have a width that is greater than the thickness of the first and second longitudinal stops.

According to another example (“Example 28”), the anchoring structure of Example 27, further comprising a tubular graft material, wherein the tubular graft materials extends through the lumen of the tubular body, everts over the second end of tubular body, and is positioned adjacent to the outer surface of the wall of the tubular body between the tubular body and the first and second longitudinal stops.

According to another example (“Example 29”), the anchoring structure of any of the preceding Examples, the arms are covered in fabric, either individually or in aggregate.

According to another example (“Example 30”), the anchoring structure of Example 29, the fabric material comprises ePTFE.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of an example cannula and anchoring structure, in accordance with various aspects of the present disclosure.

FIG. 2 is an illustration of an example anchoring structure, in accordance with various aspects of the present disclosure.

FIG. 3 is a representation of an example cannula and anchoring structure with the struts in a deployed configuration, in accordance with various aspects of the present disclosure.

FIG. 4 is a representation of an example cannula and anchoring structure with the struts in a deployed configuration viewed from an end of the anchoring structure, in accordance with various aspects of the present disclosure.

FIG. 5 is a representation of an example anchoring structure positioned in an exemplary delivery device, in accordance with various aspects of the present disclosure.

FIGS. 6A-6C is a representation of an exemplary delivery method of an example anchoring structure being deployed from an example delivery device, in accordance with various aspects of the present disclosure.

FIG. 7 is an illustration of an example anchoring structure include apertures formed through walls of the anchoring structure, in accordance with various aspects of the present disclosure.

FIG. 8 is an illustration of an example anchoring structure with a second end that is fluted, in accordance with various aspects of the present disclosure.

FIGS. 9A-9D are illustrations of an example anchoring structure with a plurality of struts extending through a wall of a tubular body, in accordance with various aspects of the present disclosure.

FIGS. 10A-10D are illustration of an example anchoring structure with longitudinal stops including a ring and struts extending from the ring, in accordance with various aspects of the present disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed to apparatuses, systems, and methods that include a cannula. The cannula may be arranged within a patient and create a fluid passageway. The cannula may extend from an organ (e.g., vasculature, heart, gallbladder, liver, or lungs) and connect to a device, another organ, or exit the patient. In certain instances, the cannula may be a graft or stent graft that is arranged within the vasculature. The apparatuses, systems, and methods may be used in improving or assisting the vascular and/or cardiac function of the heart. The cannula, as discussed in further detail below, may include an anchoring structure. The anchoring structure may be arranged to anchor with a tissue between two compartments (e.g., two vessels, heart chambers, or other bodily structure) within a patient. The anchoring structure may be arranged between vessels or between different portions of the same vessel, or on either side of a septum within a patient's heart, for example. The disclosed anchoring structure or inlet portion of a cannula may be used with a pump device or system, such as a vascular pump or cardiac assist device for managing (e.g., increasing) blood flow in a patient.

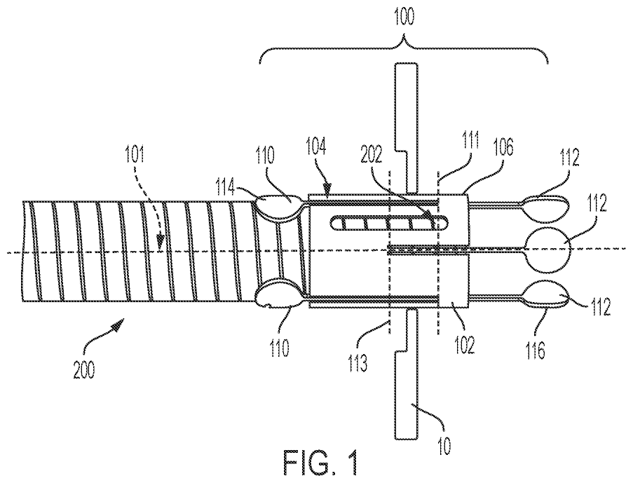

FIG. 1 is an illustration of an example cannula 200 arranged within a patient's heart 10, or other bodily structure 10 (e.g., vasculature, organs, or other bodily structure), in accordance with various aspects of the present disclosure. The cannula 200 includes an anchoring structure 100 and may be used for an inflow or outflow cannula. The anchoring structure 100 is arranged at or adjacent to an end of a conduit 202 of the cannula 200. The conduit 202 may be configured to provide a fluid passageway from a target location or between two target locations.

Any number of cannulas may be implemented, and the disclosure is not to be limiting on the scope of the cannula. In one example, the cannula 200 is formed of a graft structure that may include reinforcing elements such as rings or a helical structure (e.g., helically wound wire or a tube with a helical cut extending around and longitudinally along the length of the cannula 200). In some embodiments, the cannula 200 may include a coating or material (e.g., a graft material such as, but not limited to ePTFE) for forming a fluid impermeable barrier for the cannula 200.

As illustrated in FIG. 1, the cannula 200 may include a first end 202 that is positioned within at least a portion of the anchoring structure 100 and define a longitudinal axis 101. For example, the anchoring structure 100 includes a tubular body 102 including a first end 104, a second end 106, and a lumen 108 configured to receive the first end 202 of the cannula 200 within the tubular body 102. By positioning the cannula 200 at least partially within the lumen 108 of the tubular body 102, kinking of the cannula 200 is reduced as the tubular body 102 of the anchoring structure 100 acts to reinforce the cannula 200. The tubular body may be affixed to the anchoring structure via welding, brazing, adhesive potting, or other method.

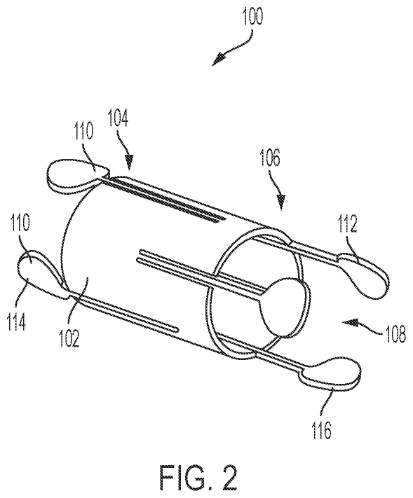

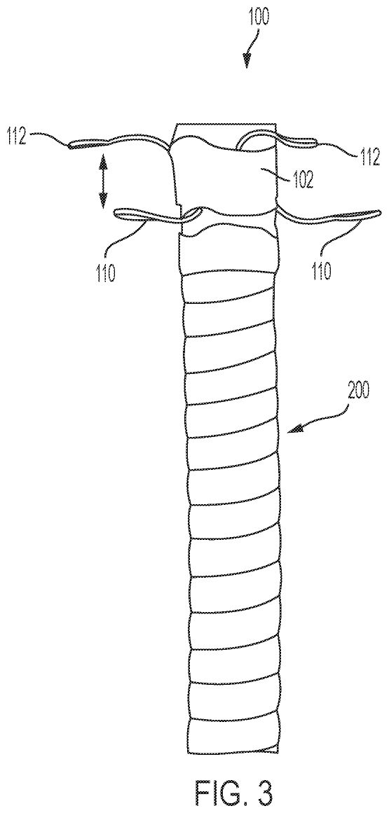

Referring to FIGS. 1 and 2, the anchoring structure 100 further includes a first plurality of struts 110 and a second plurality of struts 112. In a deployed and/or unconstrained state, the first and second plurality of struts 110, 112 extend from the tubular body 102 (e.g., in a generally radial direction as shown) and are operable to engage tissue of a patient (e.g., cardiac tissue, vascular tissue, and so forth) when positioned in the deployed configuration (see FIG. 3). More specifically, each of the plurality of struts extends from the tubular body 102 at a position between the first end 104 and the second end 106 of the tubular body. In some embodiments, the first plurality of struts 110 extend from the tubular body 102 at a first longitudinal position 111 that is between the first and second ends 104, 106 and the second plurality of struts 112 extends from the tubular body at a second longitudinal position 113 between the first and second ends 104, 106. The first and second longitudinal positions 111, 113 are spaced from each other. The first plurality of struts 110 may extend (e.g., radially) from the tubular body 102 at the first longitudinal position 111 that is between the first and second ends 102, 104, and the second plurality of struts 112 extends from the second longitudinal position 113, wherein the first longitudinal position 111 is between the second end 106 and the second longitudinal position 113 and the second longitudinal position 113 is between the first longitudinal position 111 and the first end 104. For example, the first plurality of struts 110 may extend from adjacent the second end 106 of the tubular body 102 toward the first end 104 of the tubular body 102 and the second plurality of struts 112 extend from a midpoint of the tubular body 102. In other embodiments (not shown), the first longitudinal position 111 is between the first end 104 and the second longitudinal position 113 and the second longitudinal position 113 is between the first longitudinal position 111 and the second end 106. Stated otherwise, when in the delivery configuration, the first and second plurality of struts 110, 112 include have portions that are longitudinally overlapping along the tubular body 102.

Each of the plurality of struts 110, 112 may include a pad (e.g., a first pad 114 for each of the first plurality of struts 110 and a second pad 116 for each of the second plurality of struts 112. The first and second pads 114, 116 may be positioned extending from the ends of the first and second plurality of struts 110, 112, respectively. The pads 114, 116 are operable to engage with the patient's tissue when the anchoring structure 100 is in the deployed configuration. The pads 114, 116 are shaped to include an increased surface area (e.g., to help distribute pressure applied to the patient's tissue to reduce or prevent trauma to the tissue. For example, the pads 114, 116 may include a circular, ovular, or otherwise widened profile (e.g., relative to the first and second pluralities of struts 110, 112) to increase the surface are of the strut 110, 112 at the pads 114, 116. In some embodiments, the pads 114, 116 may be positioned in the delivery configuration such that the pads 114, 116 are flush with an outer surface of the tubular body 102 (see FIGS. 1 and 2). In some embodiments, the pads may be covered with fabric, individually or in aggregate, to improve healing and mechanical stability of the implant.

In some embodiments, when in the delivery configuration (e.g., constrained and delivery through an endoluminal device that may include a delivery catheter and/or introducer sheath), the first plurality of struts 110 are configured to extend longitudinally from the tubular body 102 and toward the second end 106 of the tubular body 102 and the second plurality of struts 112 are configured to extend longitudinally from the tubular body 102 and toward the first end 104 of the tubular body 102. For example, the first plurality of struts 110 may extend from adjacent the second end 106 of the tubular body 102 toward the first end 104 of the tubular body and the second plurality of struts 112 extend from a midpoint of the tubular body 102. The first pad 114 of each of the first plurality of struts 110 extends a first distance beyond the first end 104 of the tubular body 102 and the second pad 116 of each of the second plurality of struts 112 extends a second distance that is greater than the first distance beyond the second end 106 of the tubular body 102.

Referring to FIG. 3, the anchoring structure 100 includes a deployed configuration in which the first plurality of struts 110 and the second plurality of struts 112 deflect or are deflected away from the tubular body 102. As the first and second plurality of struts 110, 112 transition to the deployed configuration, the first and second pads 114, 116 are operable to transition radially outward and longitudinally inward. For example, the first pad 114 of each of the first plurality of struts 110 transitions radially outward and longitudinally from the first end 104 toward the second end 106 of the tubular body 102. Likewise, the second pad 116 of each of the second plurality of struts 112 are configured to transition radially outward from the second end 106 toward the first end 104 of the tubular body 102.

The curvature of the first and second plurality of struts 110, 112 may be gradual so as to reduce strain on the struts when engaged with tissue and/or during delivery. As the plurality of struts 110, 112 transition from the delivery configuration to the deployed configuration, the pads 114, 116 are operable to contact the tissue (e.g., opposing surfaces of the tissue) of the patient, such that the tissue is positioned between the first pads 114, and the second pads 116. As shown in FIG. 3, in the delivery configuration, the pads 114, 116 are shown longitudinally spaced from each other to define a gap, or space between the first and second pads 114, 116 to receive tissue that may be engaged by the first and second pads 114, 116. The longitudinal spacing of the pads 114, 116 may be adjusted to specific tissue thicknesses and sensitivity of a specific tissue to pressure (e.g., softer tissues, damaged tissues, and so forth). The longitudinal spacing of the pads 114, 116 may be reduced when tighter anchoring is desired (e.g., greater pressure is to be applied to the tissue interposed between the pads 114, 116), and in some embodiments there may be no longitudinal spacing between the pads 114, 116 or the planes of the tissue contacting surface of the pads 114, 116 is negative (e.g., the planes of the surfaces longitudinally pass each other as the plurality of struts 110, 112 are deployed).

As previously described, the plurality of struts 110, 112 may include a curved profile without bends, folds, or creases in order to reduce strain on the struts, and which is also operable to position the pads 114, 116 to be longitudinally spaced from each other when in the deployed configuration. The curved profile may include a first bend in a first direction and a second bend in a second direction. For example, a first portion of each strut 110, 112 may be curved away from the tubular body 102. The first portion may curve through greater than 90 degrees and less than 180 degrees. The second portion of each strut may curve in the second direction a complementary amount to orient the pads 114, 116 at about 90 degrees relative to the outer surface of the tubular body 102. The pads 114, 116 may be spaced from about 2 mm to about 15 mm (e.g., about 5 mm between the pads 114, 116).

As illustrated in FIG. 3, in some embodiments, when the plurality of struts 110, 112 are positioned in the delivery configuration, the tubular body 102 extends longitudinally beyond the furthest longitudinal extents, or the ends, of the plurality of struts 110, 112. Stated otherwise, the plurality of struts 110, 112 evert back in the deployed configuration such that the plurality of struts 110, 112 are positioned longitudinally between the first and second ends 104, 106 of the tubular body 102 when in the deployed configuration. For example, second end 106 of the tubular body 102 may longitudinally extend about 1.5 mm beyond the furthest longitudinal extent of the second plurality of struts 112 when in the deployed configuration. Because the plurality of struts 110, 112 are not longitudinally centered on the midline of the tubular body 102, the first end 104 may longitudinally extend more than 1.5 mm beyond the furthest longitudinal extent of the first plurality of struts 110 when in the deployed configuration.

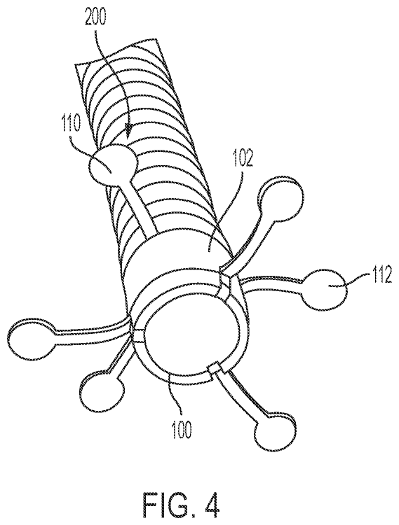

FIG. 4 provides a representation of the cannula 200 and anchoring structure 100 with the struts 110, 112 in a deployed configuration viewed from the second end 106 of the anchoring structure 100. In some embodiments, the first plurality of struts 110 are circumferentially staggered relative to the second plurality of struts 112 along the tubular body 102. In those embodiments in which the first plurality of struts 110 includes three struts and the second plurality of struts 112 includes three struts, the struts of the first plurality of struts 110 are evenly spaced relative to each other (e.g. 120 degrees) and the second plurality of struts 112 are evenly spaced relative to each other. The first and second plurality of struts 110, 112 are offset from each other such that each strut of the second plurality of struts 112 is spaced 60 degrees relative to adjacent struts of the first plurality of struts 110, and vice versa. It is noted that any number of struts may be implemented with respect to the first and/or second plurality of struts 110, 112 and that the struts of each of the pluralities of struts 110, 112 may be circumferentially offset from each other at various angles. By circumferentially offsetting the struts of the first and second plurality of struts 110, 112, the localized forces applied to the tissue may be minimized, or in different terms, the forces applied to the tissue may be more desirably distributed, and increased traction and sealing between the anchoring structure 100 may be achieved. Furthermore, the circumferential offsetting of the struts also enables portions of the first and second plurality of struts 110, 112 to longitudinally overlap along the length of the tubular body 102.

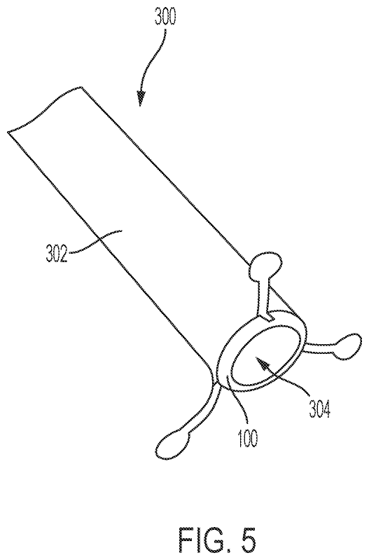

Referring to FIG. 5, an anchoring structure 100 is positioned partially in a delivery device 300. For example, the delivery device 300 may include a tubular element 302 that includes a central lumen 304 within which the anchoring structure 100 may be positioned and constrained in the delivery configuration.

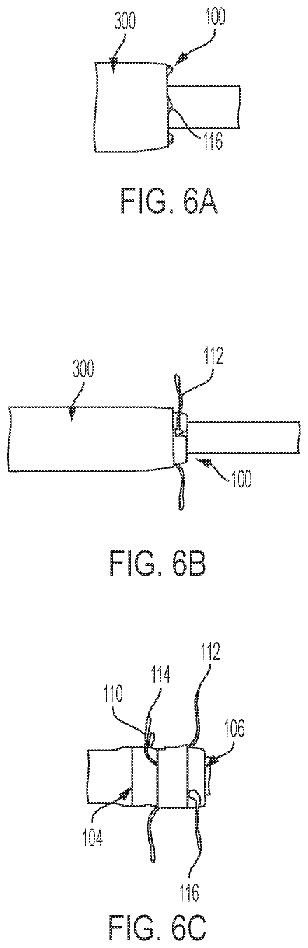

FIGS. 6A-6C demonstrate the anchoring structure 100 being deployed from the delivery device 300. For example, FIG. 6A shows a delivery device 300 surrounding the anchoring structure 100 (the tips of the pads 116 are visible in FIG. 6A). The delivery device 300 can be retracted from around the anchoring structure such that the second plurality of struts 112 may be released from the delivery device 300. In those embodiments in which the anchoring structure and/or the plurality of struts 110, 112 are formed of a shape memory material, the plurality of struts 110, 112 may be shape set to automatically transition to the deployed configuration when the plurality of struts 110, 112 are released from (e.g., no longer constrained by) the delivery device 300. For example, FIG. 6B demonstrates the second plurality of struts 112 being released from the delivery device 300 and which transition longitudinally and radially to a deployed configuration. FIG. 6C demonstrates the first plurality of struts 100 being released from the delivery device 300 and which transition longitudinally and radially to a deployed configuration. For example, the first pad 114 of each of the first plurality of struts 110 is operable to longitudinally transition toward the second end 106 of the tubular body 102 and the second pad 116 of each of the second plurality of struts 112 is operable to transition toward the first end 104 of the tubular body 102 when the anchoring structure 100 is transitioned toward the deployed configuration. Furthermore, the first pad 114 of each of the first plurality of struts 110 and the second pad 116 of each of the second plurality of struts 112 is operable to transition radially outward when the anchoring structure 100 is transitioned toward the deployed configuration. It is noted that the anchoring structure 100 may be formed from a cut tube (e.g., laser cut) and the plurality of struts 110, 112 may be shape set to the deployed configuration such that the anchoring structure has a tubular profile in the delivery configuration and the struts expand radially outward in the deployed configuration. The plurality of struts 110, 112 are thus formed from portions of a wall 103 forming the tubular body 102.

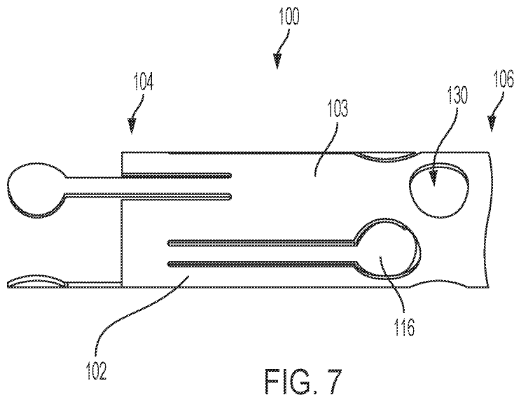

Referring now to FIG. 7, an anchoring structure 100 include apertures 130 formed through walls 103 of the tubular body 102. The apertures may include, for example, circular apertures positioned at the second end 106 of the tubular body 102 or slots that extend longitudinally along at least a portion of the tubular body (see FIG. 1). The apertures 130 are operable to provide improved flow characteristics of fluids flowing through the anchoring structure 100 and the cannula 200. The additional apertures serve to reduce fluid velocity and shear on entry into the cannula. The additional apertures also provide redundant fluid entry sites should the cannula tip become sealed against tissue.

In some embodiments, the at least one set of the first plurality or second plurality of struts 110, 112 may be longitudinally positioned between the first end 104 and second end 106 of the tubular body 102 when in the delivery configuration. In this embodiment, the second pads 116, for example, may be cut from the tubular body 102 such that the second end 106 of the tubular body 102 extends beyond the second pads 116 when in the delivery configuration.

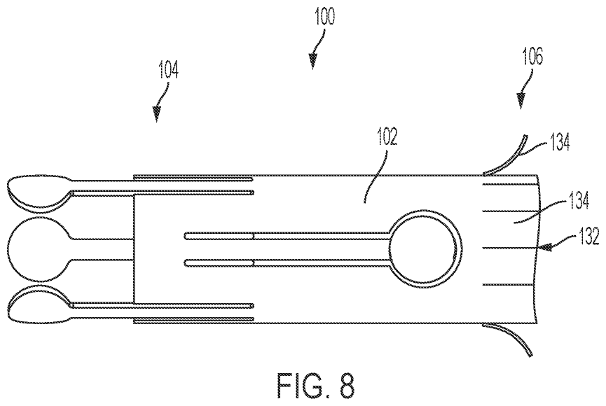

FIG. 8 provides an example of an anchoring structure 100 having a second end 106 that is fluted. For example, slits 132 may be formed longitudinally through the first and/or second end 104, 106 of the tubular body 102. The slits 132 define flaps 134 that are operable to flare radially outwardly to form the fluted end. The fluted end may further contribute to improved fluid flow characteristics by reducing fluid velocity and shear. Any number of these features may be used in combination such as apertures 130, fluted ends, various positions of the plurality of struts 110, 112, and so forth.

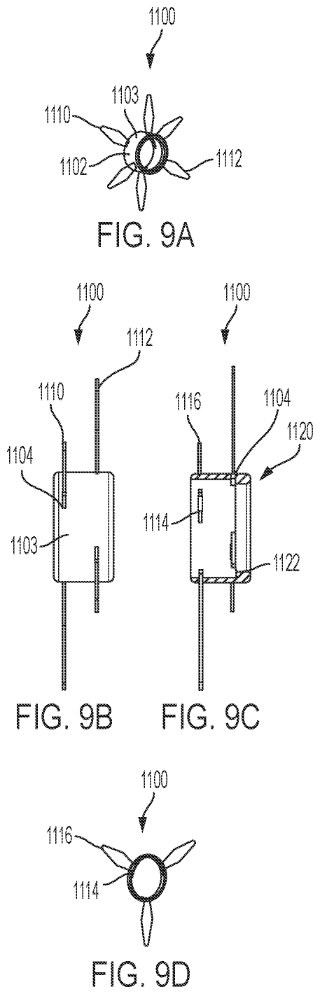

Referring to FIGS. 9A-9D an example anchoring structure 1100 with a plurality of arms 1110, 1112 extending through a wall 1103 of a tubular body 1102 are illustrated. In this embodiment, the tubular body 1102 may include a screw barrel that is formed with apertures 1104 through the wall 1103 through which the plurality of arms 1110, 1112 extend. In some embodiments the tubular body 1102 may include a first end interior diameter that is greater than a second end interior diameter. For example, the second end 1120 of the tubular body 1102 may include a lip 1122. The lip 1122 may be configured to create turbulence to flow to lessen potential thrombus formation.

In some embodiments, the first plurality of arms 1110 may be positioned at a first longitudinal position along the tubular body 1102 and the second plurality of arms 1112 may be positioned at a second longitudinal position along the tubular body 1102. The first and second plurality of arms 1110, 1112 may each be formed of a base portion 1114 and a body portion 1116. In various examples, the base portion 116 is coupled to the wall 1103 such that each of the pluralities of arms 1110, 1112 extend radially from the tubular body 1102. The first plurality of arms 1110 and the second plurality of arms 1112 are arranged to define a gap therebetween for receiving tissue. In terms of an optional coupling mechanism, in some examples, the base portion 1114 is positioned on an interior side of the wall 1103 of the tubular body 1102. The base portion 1114 is formed such that it contacts the interior surface 1124 of the wall 1103 and provides resistance to radial movement of the arms 1110, 1112 relative to the tubular body 1102 (see FIG. 9C). The body portion 1116 of the arms 1110, 1112 extends through the apertures 1104 in the wall 1103 of the tubular body 1102 (see FIG. 9D). The arms 1110, 1112 extend radially outward from the tubular body 1102 (see FIG. 9B). The first plurality of arms 1110 may be circumferentially offset from the second plurality of arms 1112, similar to the circumferential offset of the plurality of struts 110, 112 as previously discussed (see FIG. 9A). The arms 1110, 1112 may be flared at a mid-portion relative to the radial ends of the arms, thus increasing the surface area for contacting the tissue at a midportion of the arms 1110, 1112. For example, the arms 1110, 1112 may have a parallelogram shape. It is contemplated that any suitable shape of the arms 1110, 1112 may be implemented in accordance with the anchoring structure 1100, including but not limited to, straight, petaloid, diamond, and so forth. The arms 1110, 1112 may be formed of wire.

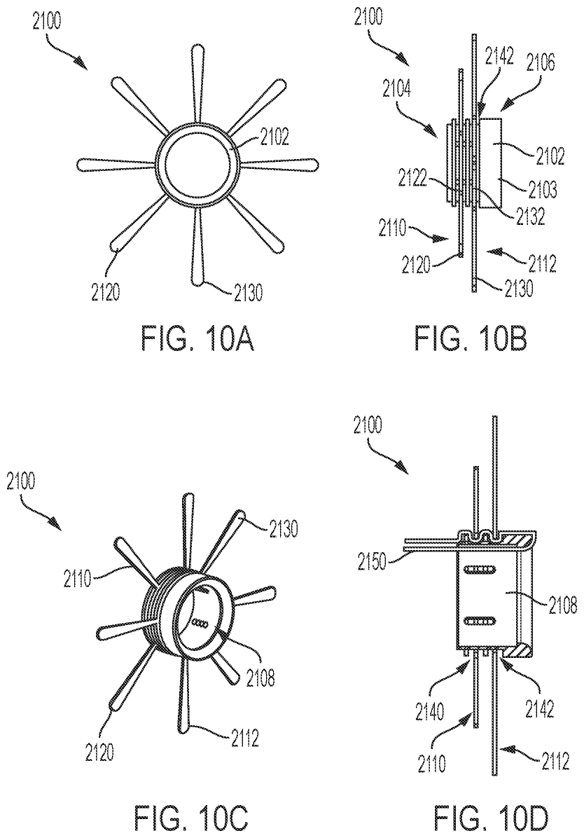

Referring now to FIGS. 10A-10D, an illustration of an anchoring structure 2100 a tubular body 2102 having a wall 2103 including a first end 2104 and a second end 2106 and longitudinal stops 2110, 2112. The tubular body 2102 includes the wall 2103 having a lumen 2108 defined from the first end 2104 to the second end 2106. The first longitudinal stop 2110 includes a first plurality of arms 2120 circumferentially spaced and extending radially outward from a first ring 2122 engaged with the tubular body 2102 at a first longitudinal position. The second longitudinal stop 2112 includes a second plurality of arms 2130 circumferentially spaced and extending radially outward from a second ring 2132 engaged with the tubular body 2102 at a second longitudinal position. The second plurality of arms 2130 may be circumferentially offset relative to the first plurality of arms 2120.

In some embodiments, the first plurality of arms 2120 has a first arm length and the second plurality of arms 2130 has a second arm length, the first arm length being less than the second arm length. It is understood that any of the aforementioned embodiments may implement arms or struts that have different length or may be of uniform length. In some embodiments, each of the arms of the first plurality and second plurality of arms 2120, 2130 is wider at a tip than at a base.

The tubular body 2102 may include circumferential slots 2140, 2142 for engaging the longitudinal stops 2110. For example, the tubular body 2102 may include a first circumferential slot 2140 formed in an outer surface of the wall 2103 and a second circumferential slot 2142 formed in the outer surface of the wall 2103 longitudinally spaced from the first circumferential slot 2140. The first ring 2122 of the first longitudinal stop 2110 may be positioned in the first circumferential slot 2140 and the second ring 2132 may be positioned in the second circumferential slot 2142. The circumferential slots 2140, 2142 define the longitudinal lengths at which the longitudinal stops 2110, 2112 are positioned. In some embodiments, the first and second longitudinal stops 2110, 2112 have a thickness and the first and second circumferential slots 2140, 2142 each have a width that is greater than the thickness of the first and second longitudinal stops 2110, 2112. In this embodiment, a tubular graft material 2150 may be provided with the anchoring structure 2100, wherein the tubular graft material 2150 extends through the lumen 2108 of the tubular body 2102, everts over the second end 2106 of tubular body 2102, and is positioned adjacent to the outer surface of the wall 2103 of the tubular body 2102 between the tubular body 2102 and the first and second longitudinal stops 2110, 2112. In these embodiments, the tubular graft material 2150 is clamped by the longitudinal stops 2110, 2112 to the outer surface of the tubular body 2102. Other devices discussed herein may also be incorporated with the tubular graft material 2150 by following a similar path. The continuous tubular graft material provides a homogenous surface which may reduce laminated or bonded edges, in turn limiting the potential for thrombus at the cannula inlet.

A biocompatible material for the graft components, discussed herein, may be used. In certain instances, the graft may include a fluoropolymer, such as a polytetrafluoroethylene (PTFE) polymer or an expanded polytetrafluoroethylene (ePTFE) polymer. In some instances, the graft may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

In addition, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

In certain instances, the coating, as discussed in detail above, may include bio-active agents in addition to heparin or alternatively to heparin. The agents can include, but are not limited to, vasodilator, anti-coagulants, anti-platelet, anti-thrombogenic agents.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A cannula comprising:
a conduit having an exterior surface, an interior surface, a first end, and a second end; and
an anchoring structure arranged at the first end of the conduit, the anchoring structure including:
a tubular body including a first end, a second end, and a lumen configured to receive the end of the conduit within the tubular body,
a first plurality of struts extending from a first longitudinal position between the first and second ends of the tubular body or from adjacent the second end of the tubular body toward the first end of the tubular body with each of the first plurality of struts including a first pad, and
a second plurality of struts of struts extending from a second longitudinal position that is the same or different from the first longitudinal position and is between the first and second ends of the tubular body or extending from adjacent the first end of the tubular body toward the second end of the tubular body with each of the second plurality of struts including a second pad wherein the first and second plurality of struts cross one another longitudinally.

2. The cannula of claim 1, wherein the first plurality of struts extends from adjacent the second end of the tubular body toward the first end of the tubular body and the second plurality of struts extend from a midpoint of the tubular body.

3. The cannula of claim 1, wherein the first pad of each of the first plurality of struts extends a first distance beyond the first end of the tubular body and the second pad of each of the second plurality of struts extends a second distance that is greater than the first distance beyond the second end of the tubular body in a delivery configuration.

4. The cannula of claim 1, wherein the first plurality of struts are configured to extend longitudinally from the tubular body and toward the second end of the tubular body and the second plurality of struts are configured to extend longitudinally from the tubular body and toward the first end of the tubular body in a deployed configuration.

5. The cannula of claim 4, wherein the first plurality of struts are longitudinally spaced from the second plurality of struts along a longitudinal axis of the tubular body in the deployed configuration.

6. The cannula of claim 4, wherein the first pad of each of the first plurality of struts and the second pad of each of the second plurality of struts are configured to contact opposing surfaces of a tissue wall in the deployed configuration.

7. The cannula of claim 1, wherein the first plurality of struts are circumferentially staggered relative to the second plurality of struts along the tubular body.

8. The cannula of claim 1, wherein the first pad of each of the first plurality of struts is operable to longitudinally transition toward the second end of the tubular body and the second pad of each of the second plurality of struts is operable to longitudinally transition toward the first end of the tubular body when the anchoring structure is transitioned toward the deployed configuration.

9. The cannula of claim 1, wherein the first pad of each of the first plurality of struts and the second pad of each of the second plurality of struts is operable to transition radially outward when the anchoring structure is transitioned toward the deployed configuration.

10. The cannula of claim 1, wherein the first plurality of struts and the second plurality of struts include a curved profile when in the deployed configuration.

11. The cannula of claim 10, wherein the curved profile includes a first portion that is curved in a first direction and a second portion that is curved in a second direction.

12. The cannula of claim 11, wherein the first portion of the curved profile is greater than 90 degrees.

13. The cannula of claim 1, wherein the first plurality of struts are operable to be deployed prior to the second plurality of struts.

14. The cannula of claim 1, wherein the first plurality of struts and the second plurality of struts are shape set to the deployed configuration.

15. The cannula of claim 1, wherein the tubular body includes a wall including apertures formed through the wall.

16. The cannula of claim 1, wherein the first end of the tubular body is fluted.

17. An anchoring structure of a cannula, comprising:
a tubular body having a wall including a first end and a second end, the wall having a lumen defined from the first end to the second;
a first plurality of arms circumferentially spaced and extending radially outward from the tubular body at a first longitudinal position, each arm of the first plurality of arms including a base portion positioned within the lumen of the tubular body and a body portion extending through and beyond the wall of the tubular body; and
a second plurality of arms circumferentially spaced and extending radially outward from the tubular body at a second longitudinal position, each arm of the second plurality of arms including a base portion positioned within the lumen of the tubular body and a body portion extending through and beyond the wall of the tubular body, the second plurality of arms being circumferentially offset relative to the first plurality of arms.

18.-22. (canceled)

23. An anchoring structure of a cannula, comprising:
a tubular body having a wall including a first end and a second end, the wall having a lumen defined from the first end to the second;
a first longitudinal stop including a first plurality of arms circumferentially spaced and extending radially outward from a first ring engaged with the tubular body at a first longitudinal position; and
a second longitudinal stop including a second plurality of arms circumferentially spaced and extending radially outward from a second ring engaged with the tubular body at a second longitudinal position, the second plurality of arms being circumferentially offset relative to the first plurality of arms.

24.-30. (canceled)
